(19) United States
(12) Patent Application Publication
(10) Pub. No.: US 2023/0119968 A1
(43) Pub. Date: Apr. 20, 2023

(54) Processes for Breaching Blood-Brain Barrier

(71) Applicant: Walter A. Tormasi, Martinsville, NJ (US)

(72) Inventor: Walter A. Tormasi, Martinsville, NJ (US)

(21) Appl. No.: 17/803,470

(22) Filed: Jul. 28, 2022

Related U.S. Application Data
(60) Provisional application No. 63/360,616, filed on Oct. 18, 2021.

(51) Int. Cl. A61K 33/00; A61K 9/00

(52) U.S. Cl. CPC A61K 33/00; A61K 9/007; A61K 9/0085; A61K 2121/00

(57) ABSTRACT

Disclosed are various methods and systems for increasing the permeability of cerebral capillaries. Increased permeability is accomplished via stretching means, inflaming means, overtaxing means, underoxygenating means, and/or immunocompromising means. Preferred stretching means include vasoconstrictors and cardiostimulators. Preferred inflaming means include electromagnetic fields and sonic waves. Preferred overtaxing means include xenon administration. Preferred underoxygenating means include airway-regulated oxygen deprivation. Preferred immunocompromising means include immunosuppressants and ionizing radiation. Also disclosed are processes for subsequently normalizing the permeability of cerebral capillaries. All disclosed means/processes can be selectively grouped, that is, combined or omitted, in accordance with the invention. By implementing the methods and systems invented, practitioners will be capable of opening (and closing) the blood-brain barrier, in which event therapeutic and diagnostic agents can enter the central nervous system and can act upon neurovascular-protected brain tissue.

### CROSS-REFERENCE TO RELATED APPLICATIONS

This patent claims priority to U.S. Provisional Pat. Application No. 63/360,616, said application filed by the inventor herein, Walter A. Tormasi, on 18 Oct. 2021.

### FIELD OF THE INVENTION

The invention is directed at increasing the permeability of cerebral capillaries, thus enabling therapeutic and diagnostic agents to enter the central nervous system and to act upon neurovascular-protected brain tissue. Multiple fields are implicated by the invention. The main fields, however, relate to biotechnological processes, neurobiological treatments, and intracranial drug-delivery methods and systems.

### BACKGROUND OF THE INVENTION

A plethora, of brain disorders exist. Those brain disorders range from cancer to psychosis to dementia to depression to migraines. Cancer, of course, has mortal consequences, resulting in approximately 25,000 deaths annually in the United States. Even more widespread are nonterminal brain disorders. Such disorders, though survivable, cause mental anguish and physical disabilities, negatively impacting the lives of millions of men, women, and children (some of whom commit suicide or engage in antisocial behavior).

Given their mortal, physical, psychological, and/or quality-of-life implications, brain disorders are extremely serious. This holds true not only for those afflicted but also for their friends, family, and caregivers. Prompt diagnosis and treatment of brain disorders are therefore warranted.

Despite significant advances in neuroscience and biotechnology over the years, most brain disorders remain difficult or impossible to treat. The main impediment arises from the protective nature of intracranial capillaries. To treat brain disorders, drugs must be intravenously delivered to afflicted brain tissue. Cerebral capillaries, however, are tasked with protecting the central nervous system from pathogens, toxins, and other unfamiliar or dangerous foreign substances. Cerebral capillaries are therefore highly resistant to diffusion, preventing most neurotherapeutics and neurodiagnostics from reaching diseased brain tissue.

The neurovascular membrane that protects the central nervous system is known as the blood-brain barrier. The blood-brain barrier (commonly abbreviated BBB) results from the unique structure and physiology of cerebral capillaries.

Just like noncranial capillaries, cerebral capillaries are composed of endothelial cells (said endothelial cells represented in FIGS. 1 and 2 as element 1). The endothelial cells surround the capillary and serve as the capillary wall. The capillary wall/membrane, known as the endothelium, is cytoplasm-thin. The capillary wall, in other words, has the thickness of only one endothelial cell.

Unlike noncranial capillaries, however, cerebral capillaries feature special cellular interconnections. Those interconnections, which comprise transmembrane proteins, are known as tight junctions. As implied by that moniker, tight junctions closely, and tightly, join individual endothelial cells. Without such tight junctions, endothelial cells would be loose-floating (and thus permeable), allowing indiscriminate passage of substances into the central nervous system.

Most penetrations of the blood-brain barrier occur transjunctionally. That is, nearly all barrier-diffusible substances traverse the capillary extracellularly by passing through the tight junctions. The tight junctions, in that respect, can be regarded as the path of least resistance.

For completeness, it should be noted that cerebral capillaries also feature encapsulating pericytes and astrocyte endfeet (said pericytes and astrocyte endfeet represented in FIG. 2 as elements 2 and 3, respectively). Those perivascular cells are anchored to the outside of the capillary by the basement membrane (said basement membrane represented in FIG. 2 as element 4). The perivascular pericytes and astrocyte endfeet, although having no physical contact with the capillary wall, provide biochemical support to the endothelium. The pericytes and astrocyte endfeet are therefore capable of indirectly regulating or impacting diffusion.

Despite such peripheral influences, the main impediments to barrier penetration are the endothelial cells and tight junctions. Those constituents, as noted, are chiefly responsible for barrier impermeability. Accordingly, for present purposes, it can be said that the endothelial’ cells and tight junctions constitute the blood-brain barrier.

In its homeostatic condition, the blood-brain barrier is highly selective in regulating transjunctional and transcellular penetration. The barrier allows passage of various nutrients, ions, and organic anions, as well as passage of essential macromolecules such as glucose, water, and amino acids. The barrier, on the other hand, prohibits nearly 100% of large-molecule and small-molecule therapeutic and diagnostic agents from entering the central nervous system.

Penetrating the blood-brain barrier presents numerous challenges. One challenge is that any penetration must be temporary, that is, nondestructive. This is because the permanent elimination of the blood-brain barrier will forever allow pathogens, toxins, and other nefarious substances to enter the cerebral fluid, opening the door to countless brain diseases. Another challenge is that the blood-brain barrier is resilient to manipulation or circumvention. This is because the blood-brain barrier is critical to human survival and has continuously evolved, and hardened, over millions of years.

The ability to penetrate the blood-brain barrier will, of course, have momentous implications in treating brain diseases. For that reason, substantial resources have been devoted to develop solutions. Billions of dollars have been expended. Thousands of research reports have been issued. And numerous techniques have been proposed and pursued.

The results of the foregoing efforts have been disappointing. At present, there are few, if any, efficient and effective methods and systems for penetrating the blood-brain barrier. The state of art is therefore deficient, preventing neuroagents from reaching, and treating, brain tissue.

### SUMMARY OF THE INVENTION

The invention encompasses various methods and systems for penetrating the blood-brain barrier. By implementing the methods and systems disclosed, practitioners will be capable of increasing the permeability of cerebral capillaries. Such an increase in permeability will allow therapeutic and diagnostic agents to enter the central nervous system and to act upon neurovascular-protected brain tissue.

As detailed below, five combinable and interchangeable processes have been devised to penetrate the blood-brain barrier. Those processes involve the employment of stretching means, inflaming means, overtaxing means, underoxygenating means, and immunocompromising means. Such processes, whether employed individually or collectively, comprise the methods and systems hereby invented.

Select stretching, inflaming, overtaxing, underoxygenating, and immunocompromising means are listed in FIGS. 5 through 9. Preferred stretching means include vasoconstrictors and cardiostimulators. Preferred inflaming means include electromagnetic fields and sonic waves. Preferred overtaxing means include xenon administration. Preferred underoxygenating means include airway-regulated oxygen deprivation. Last but not least, preferred immunocompromising means include immunosuppressants and ionizing radiation.

The foregoing processes are directed at inducing physical, nonphysical, or semiphysical changes to either the cerebral capillaries or the homeostatic environment. The processes, in that regard, are partly overlapping and partly complementary, allowing selective grouping thereof.

Also disclosed are various processes for normalizing the permeability of cerebral capillaries. Select normalizing processes are listed in FIG. 10. As’discussed below, preferred normalizing processes include removing the applied stimuli (that is, ceasing the induced changes), as well as administering counteracting and/or recuperative agents.

Regardless of which processes are employed, combined, or omitted, the invention succeeds in increasing the permeability of cerebral capillaries and in enabling the reinstatement of the blood-brain barrier. Drugs can therefore be delivered to afflicted brain tissue, with capillary permeability being normalized upon completion of drug delivery.

### BRIEF DESCRIPTION OF THE DRAWINGS

Ten drawings are supplied. Those drawings are supplied for purposes of comprehensibility and, accordingly, are intended to exemplify, rather than limit, the invention.

FIG. 1 depicts, in perspective view, an intracranial capillary in its normal (homeostatic) condition.

FIG. 2 depicts, in cross-sectional view, an intracranial capillary in its normal (homeostatic) condition.

FIG. 3 depicts, in perspective view, an intracranial capillary when subjected to various stresses induced by the methods and systems disclosed herein.

FIG. 4 depicts, in cross-sectional view, an intracranial capillary when subjected to various stresses induced by the methods and systems disclosed herein.

FIG. 5 depicts, in list form, processes for stretching intracranial capillaries pursuant to the invention.

FIG. 6 depicts, in list form, processes for inflaming intracranial capillaries pursuant to the invention.

FIG. 7 depicts, in list form, processes for overtaxing intracranial capillaries pursuant to the invention.

FIG. 8 depicts, in list form, processes for inducing underoxygenation pursuant to the invention.

FIG. 9 depicts, in list form, processes for inducing immunocompromization pursuant to the invention.

FIG. 10 depicts, in list form, processes for reinstating the blood-brain barrier pursuant to the invention.

Included within the foregoing drawings are various elements, namely, endothelial cell 1, pericyte 2, astrocyte endfoot 3, basement membrane 4, and neuroagent 5.

### DETAILED DESCRIPTION OF THE INVENTION

The invention, as indicated, comprises five combinable and interchangeable processes for penetrating the blood-brain barrier. Such processes involve the utilization of stretching means, inflaming means, overtaxing means, underoxygenating means, and/or immunocompromising means.

Discussed in detail below are the foregoing barrier-penetrating processes, as well as various processes for restoring the permeability of cerebral capillaries. Also discussed in detail below are the scientific theories underlying the processes invented. Although scientific theories fall outside the scope of patent protection, the theories propounded could allow skilled artisans to understand why and how the invention performs its intended function. The proffered theories are therefore mentioned for background purposes.

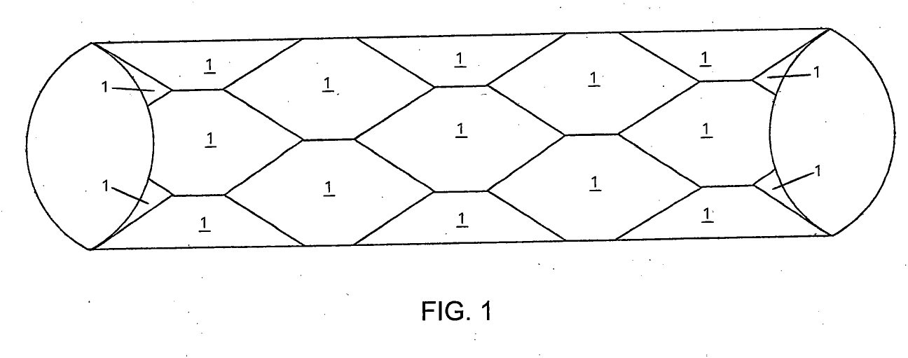

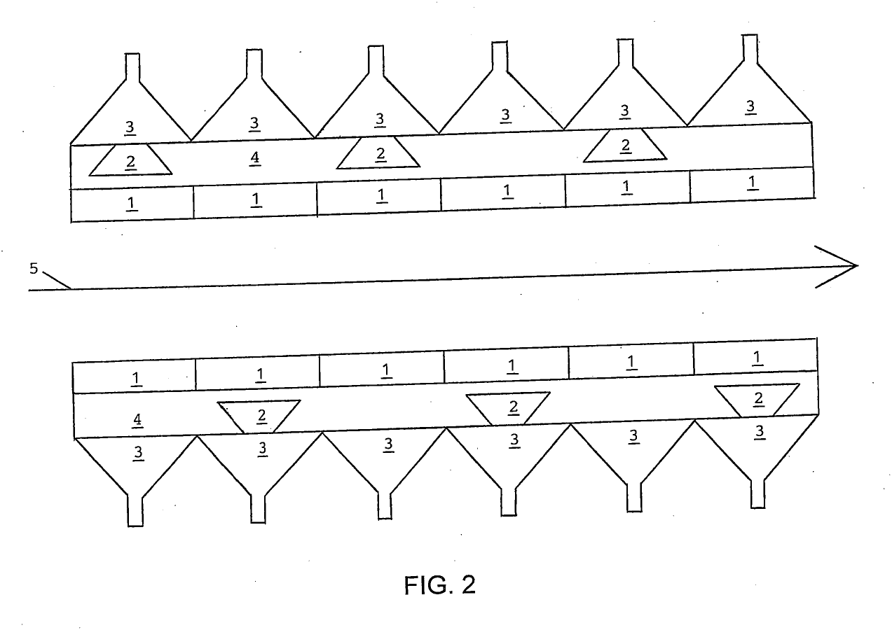

Before addressing the foregoing subjects, cerebral capillaries must be briefly discussed. FIGS. 1 and 2, as mentioned, depict an intracranial capillary in its normal physiologic condition. The capillary is composed of adjoining endothelial cells (element 1). The endothelial cells, which are elliptically shaped, feature individual nuclei (not shown). In its normal physiologic condition, known as homeostasis, the capillary is not subjected to stress and is therefore performing optimally. FIGS. 1 and 2, accordingly, show the endothelial cells (element 1) closely spaced. Such close spacing serves as an impediment to permeability, preventing intravenously administered neuroagents (represented in FIG. 2 as element 5) from passing between the tight-knitted endothelial cells.

The closely spaced endothelial cells, as depicted in FIGS. 1 and 2, constitute the blood-brain barrier. One of the primary objectives of the invention is to penetrate the blood-brain barrier, thus enabling therapeutic and diagnostic agents to enter the central nervous system and to act upon neurovascular-protected brain tissue. Various methods and systems have been devised to accomplish that objective.

As discussed below, all methods and systems seek to alter or affect the blood-brain membrane in one manner or another. Some methods and systems, for example, seek to induce physical changes to the capillary. Other methods and systems, in contrast, seek to induce nonphysical or semiphysical changes to the homeostatic environment. All induced changes, whether physical, nonphysical, or semiphysical, are transitory, allowing the blood-brain barrier to be substantially restored after neurotherapeutics or neurodiagnostics are administered.

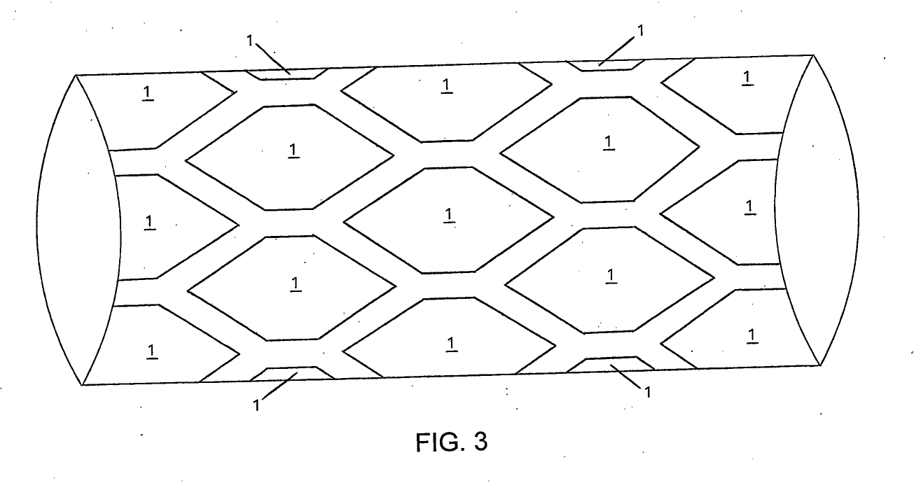

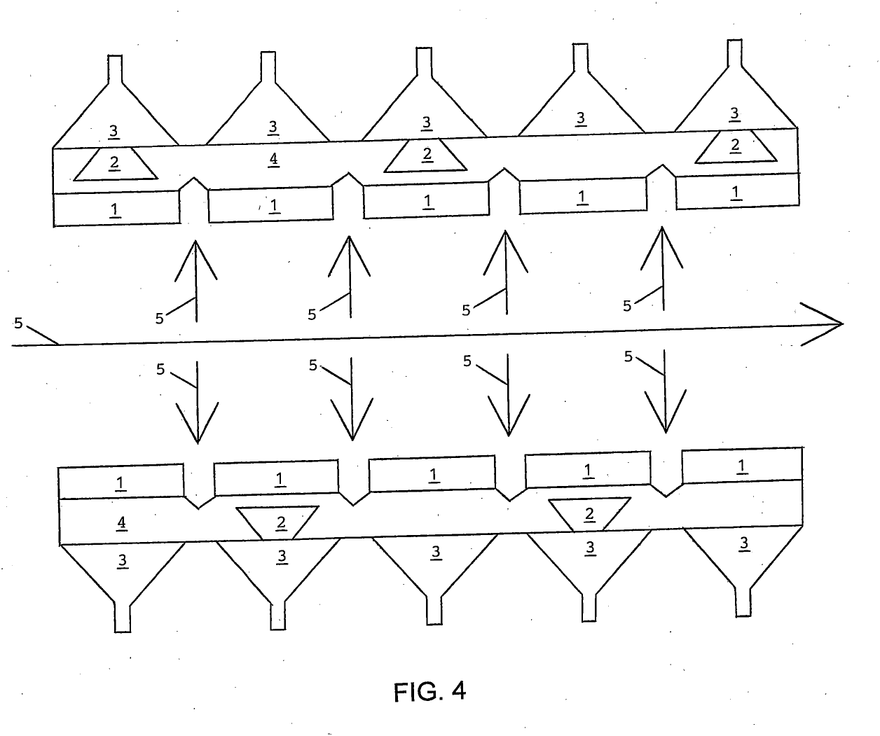

The invention, to reiterate, comprises stretching means, inflaming means,overtaxing means, underoxygenating means, and immunocompromising means. It is believed that some aspects of the invention will cause cerebral capillaries to assume the physical condition depicted in FIGS. 3 and 4. Other aspects of the invention, however, will result in nonphysical or semiphysical homeostatic changes, as noted above.

FIGS. 3 and 4 place the invention in perspective, at least in the context of the hypotheses propounded. Those drawings, in particular, depict an intracranial capillary when subjected to various stresses induced by the methods and systems disclosed. Similar to FIGS. 1 and 2, the capillary in FIGS. 3 and 4 features adjacent endothelial cells (element 1). But unlike the homeostatic capillary shown in FIGS. 1 and 2, the stress-induced capillary shown in FIGS. 3 and 4 is abnormally protracted. That physical anomaly results in the endothelial cells (element 1) being spaced farther apart. It is hypothesized that such expanded spacing causes loose/permeable intercellular junctions, as shown in FIGS. 3 and 4. It is further hypothesized that such expanded spacing allows neuroagents (represented in FIG. 4 as element 5) to pass through the blood-brain barrier transjunctionally/paracellularly.

To induce the physical and homeostatic changes intended, numerous processes have been devised. Those processes are partly overlapping and partly complementary, giving the processes redundant and synergistic effects. Such qualities allow processes to be selectively grouped, that is, combined or omitted, in accordance with the invention. Whatever group of processes is formulated and employed, those processes are effective in penetrating the blood-brain barrier and are included within the specific methods and systems claimed.

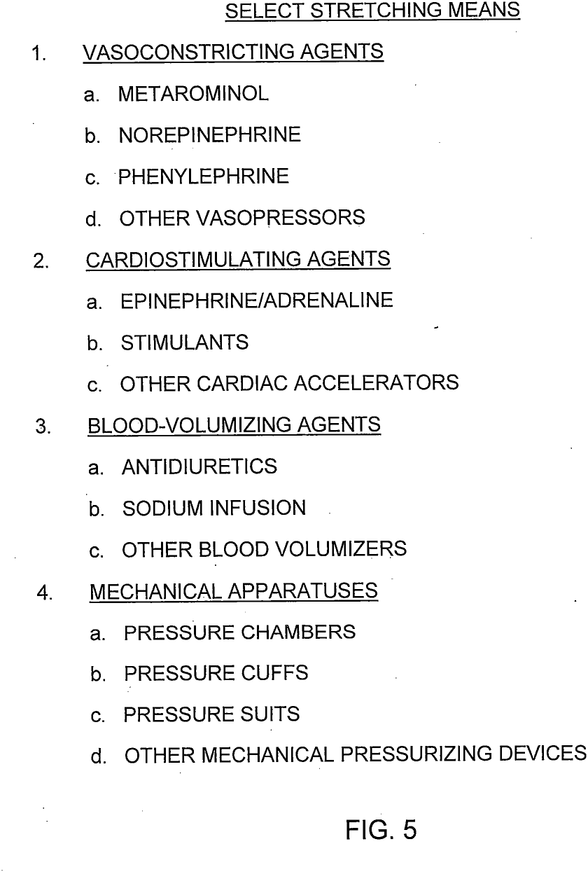

The first process involves physically stretching the cerebral capillaries. FIG. 5 depicts, in list form, various stretching means. Preferred stretching means involve raising systole or diastole blood pressure and thereby inducing intracapillary hypertension. Raising blood pressure can be accomplished by increasing either peripheral resistance or cardiac output. An increase in peripheral resistance and cardiac output can be chemically induced, respectively, via vasoconstrictors (such as norepinephrine) and cardiostimulators (such as epinephrine/adrenaline). Such chemical agents can be employed singly or jointly to raise blood pressure.

It is hypothesized that intracapillary hypertension physically stretches the cerebral capillaries. It is believed that the stretching process separates the endothelial cells and thereby loosens the intercellular junctions. The stretching and loosening processes, in turn, temporarily increase capillary permeability. The upshot is that otherwise-indiffusible substances are able to penetrate the blood-brain barrier.

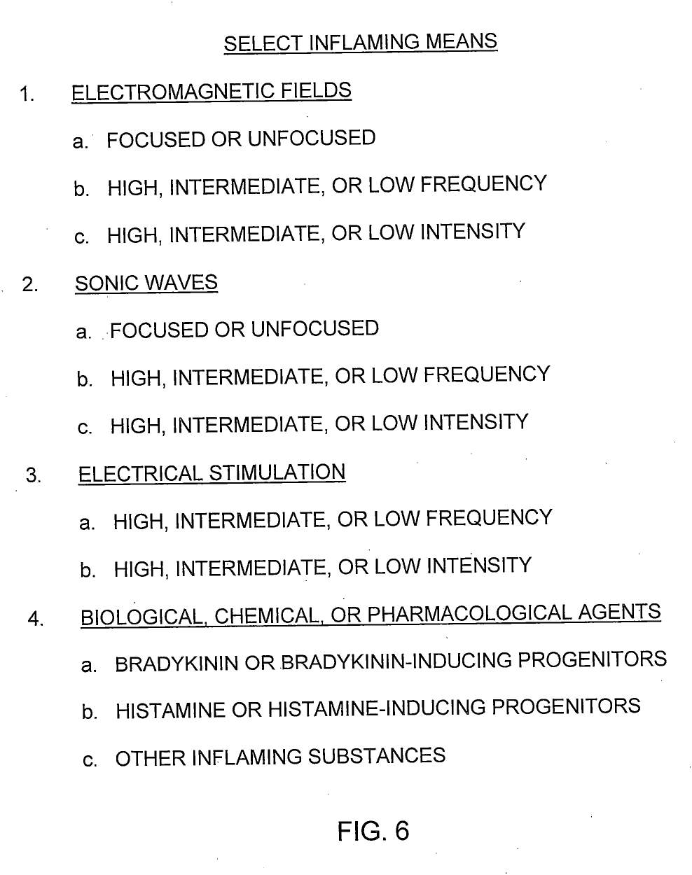

Another process involves inflaming the cerebral capillaries. FIG. 6 depicts, in list form, various inflaming means. Preferred inflaming means involve physically perturbing the cerebral capillaries using electromagnetic fields or sonic waves. Those inflaming processes can be administered extracranially, with the electromagnetic fields or sonic waves being directed at or near afflicted brain tissue. By bombarding brain tissue in that fashion, cerebral capillaries can be perturbed and agitated, resulting in inflammation.

It is hypothesized that the inflaming process expands cerebral capillaries, as depicted in FIGS. 3 and 4. It is further hypothesized that the inflaming process causes nanoscopic tears to the intercellular junctions, among other physical changes. All such physical changes will disrupt membrane function, increasing capillary permeability.

In that respect, it is known that inflammation causes swelling. Such swelling is attributed to histamine and bradykinin. Those mediator chemicals are released by the lymphatic system in response to stressive events, such as tissue injury or agitation. Capillaries, of course, constitute vascular tissue. It is therefore believed that causing capillary perturbations through electromagnetic fields or sonic waves will produce the same effect as elsewhere, namely, inflammation. The result is swelling (among other things), which causes nanoscopic tears and increases permeability.

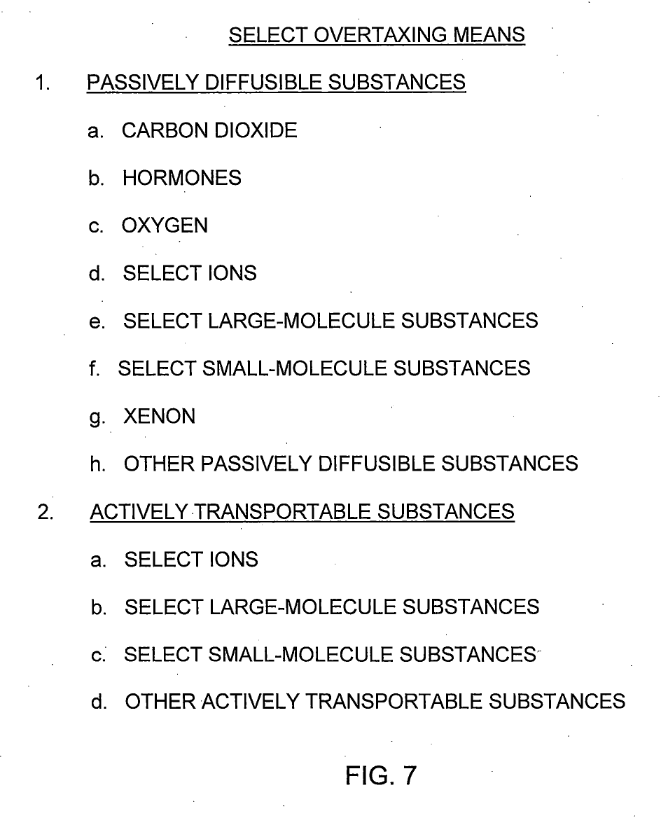

Another process for penetrating the blood-brain barrier involves overtaxing the diffusion mechanism of cerebral capillaries. FIG. 7 depicts, in list form, various overtaxing means. Preferred overtaxing means involve passing an overwhelming quantity of one or more diffusible substances through the capillary membrane. Xenon gas is particularly suitable for that purpose, given its nontoxicity and neuroprotective qualities, as well as its ability to natively traverse the blood-brain barrier. Of course, all gaseous substances, such as xenon, can be administered through respirators, ventilators, or other airway-interfacing devices.

It is hypothesized that the overtaxing process erodes the blood-brain barrier. All systems have operational parameters. Exceeding those parameters results in inefficiencies or failure. Such principles apply equally to biological systems. Accordingly, if diffusion is pushed beyond capacity (which happens when cerebral capillaries are overwhelmed by xenon or other diffusible substances), membrane decompensation ensues. The diffusion mechanism then becomes dysregulated, increasing capillary permeability.

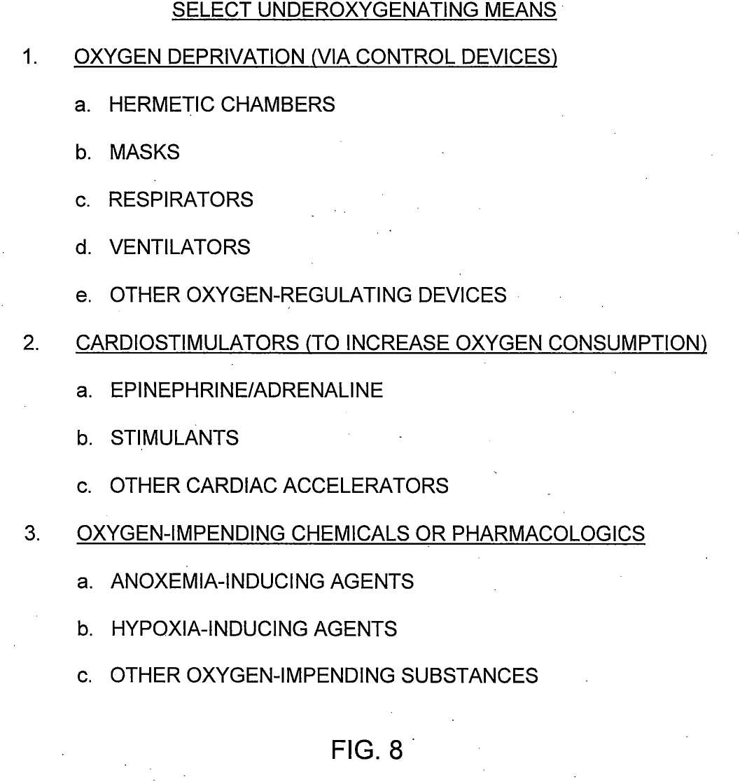

Another process for penetrating the blood-brain barrier involves underoxygenating the pulmonary/cardiovascular system. FIG. 8 depicts, in list form, various underoxygenating means. Preferred underoxygenating means involve restricting oxygen supply using airway-interfacing devices. The purpose of such oxygen restriction is to induce oxygen deficiency in the blood and tissues. It is hypothesized that inducing mild anoxemia/hypoxia will upset homeostasis, impede membrane function, and increase capillary permeability.

These homeostasis-grounded postulations cannot be lightly discounted. Human physiology is extraordinarily complex. The body encompasses numerous systems, all of which must operate in concert to keep the body alive, healthy, and protected. One homeostatic change will generate other homeostatic changes, forcing the body to compromise one system for another. Homeostatic changes, accordingly, produce cascading effects., resulting in systemic breakdown.

It goes without saying that having an adequate supply of oxygen is necessary to maintain homeostasis. For that reason, physicians have been taught to ensure that patients receive sufficient oxygen at all times. Oxygen levels are therefore closely monitored during treatment procedures, especially when patients are connected to breathing devices such as respirators or ventilators. Induced underoxygenation, needless to say, departs from conventional medical practices.

As explained above, however, homeostatic changes result in systemic breakdown. It is hypothesized that inducing slight underoxygenation will generate mild anoxemia/hypoxia, thus upsetting homeostasis. Membrane effectiveness will therefore be impeded, increasing capillary permeability.

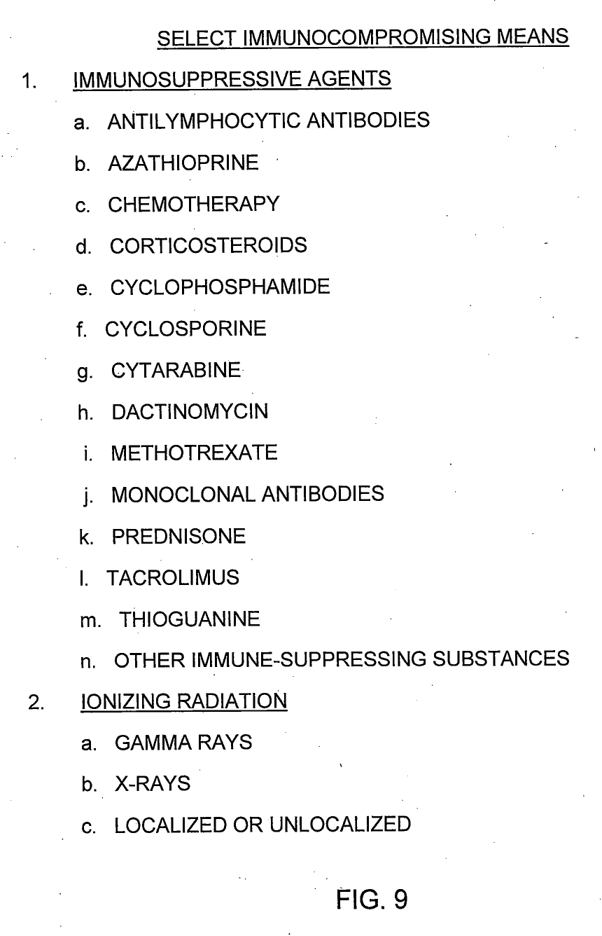

Another process for penetrating the blood-brain barrier involves immunocompromising the lymphatic system in general or local brain tissue in particular. FIG. 9 depicts, in list form, various immunocompromising means. Preferred immunocompromising means involve pharmacological agents (immunosuppressants) or, for greater targeting specificity, ionizing radiation (x-rays or gamma rays). Such immunocompromising processes are expected to dysregulate the endothelial membrane, permitting otherwise-indiffusible substances to penetrate the blood-brain barrier.

In that regard, it is hypothesized that aspects of the blood-brain barrier are influenced, at least in part, by the immune system. The immune system, for starters, is directly or indirectly tied into all other biological systems. A compromised immune system therefore upsets homeostasis, to say the least. But going beyond homeostatic changes, it is hypothesized that the immune system is essential for regulating the blood-brain barrier. Accordingly, by compromising the immune system via pharmacological agents or ionizing radiation, the blood-brain barrier can be dysregulated. Such dysregulation, in turn, increases capillary permeability.

As can be discerned from the above disclosure, the foregoing methods and systems alter or affect the blood-brain membrane in one manner or another. Some methods and systems, for example, induce physical changes to the capillary. Other methods and systems, in contrast, induce nonphysical or semiphysical changes to the homeostatic environment. These changes are not mutually exclusive, as some methods and systems cause not only physical changes to the capillary but also nonphysical or semiphysical homeostatic changes.

Advantageously, none of the above processes are expected to inflict permanent damage to the endothelial membrane, making all increases in permeability temporary. The blood-brain barrier, as such, can be restored after neurotherapeutics or neurodiagnostics are administered.

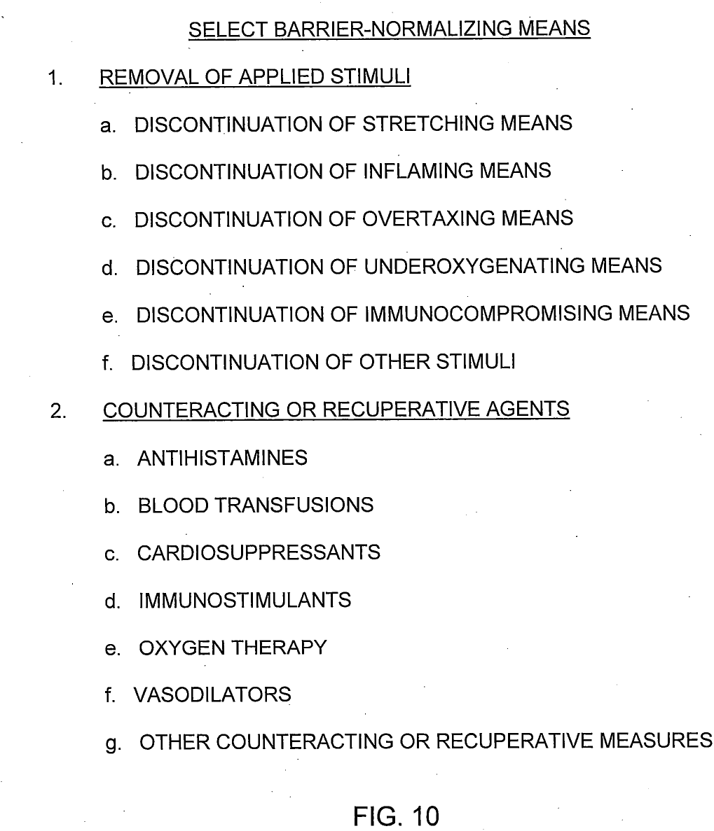

FIG. 10 depicts, in list form, various means for normalizing the permeability of cerebral capillaries. Such means can be implemented individually or in combination.

The simplest way to restore the blood-brain barrier is to remove the applied stimuli. Thus, where the preferred stretching, inflaming, overtaxing, underoxygenating, and immunocompromising means are employed, practitioners can discontinue vasoconstrictors and cardiostimulators (thus ending the stretching process); can discontinue electromagnetic or sonic bombardment (thus ending the inflaming process); can discontinue xenon diffusion (thus ending the overtaxing process); can discontinue oxygen deprivation (thus ending the underoxygenating process); and can discontinue immunosuppressants and ionizing radiation (thus ending the immunocompromising process). In most cases, removal of the applied stimuli will achieve barrier normalcy within minutes or hours, although longer restoration periods may be necessary to fully recover from the immunocompromising process.

In addition to removing the applied stimuli, certain counteracting or recuperative agents can be administered to facilitate or expedite normal barrier operation. For example, vasodilators and cardiosuppressants can be administered to reduce blood pressure (thus eliminating intracapillary hypertension), while antihistamines can be administered to reduce inflammation (thus eliminating capillary swelling and other inflammatory effects). Additional or different counteracting or recuperative agents, including those listed in FIG. 10, can be administered to lower blood pressure, reduce inflammation, bolster the immune system, reverse anoxemia/hypoxia, or otherwise achieve homeostasis.

It goes without saying that restoration of the blood-brain barrier is biologically important. This is because the permanent elimination of the blood-brain barrier will forever allow pathogens, toxins, and other nefarious substances to enter the central nervous system, opening the door to countless brain diseases. In fact, numerous neurological disorders, including Alzheimer’s disease, Parkinson’s disease, and Huntington’s disease, are associated with leaky intracranial capillaries. Because the blood-brain barrier serves protective functions, any barrier penetration must be temporary and nondestructive. The invention meets those conditions, allowing postpenetration restoration of the blood-brain barrier.

It must be reiterated that the foregoing processes are partly overlapping and partly complementary, giving the processes redundant and synergistic effects. Such qualities allow processes to be selectively grouped. Regardless of which processes are combined or omitted, all processes singly and collectively fall within the methods and systems claimed.

Throughout the present disclosure, the invention has been discussed in connection with various scientific hypotheses. Those hypotheses seek to explain why and how the invention penetrates the blood-brain barrier. For example, it has been posited that the stretching process separates the endothelial cells and loosens the intercellular junctions; that the inflaming process agitates the endothelial membrane and causes nanoscopic tears to the connective junctions; that the overtaxing process overwhelms the diffusion capacity of the intercellular junctions and disrupts conventional membrane function; that the underoxygenating process upsets the homeostatic environment and produces systemic breakdown; and that the immunocompromising process dysregulates the diffusion mechanism and increases membrane permeability.

The foregoing hypotheses certainly explain how and why the invention performs its intended function. The invention, however, is not contingent on the validity of the theories propounded. This is because the invention encompasses the methods and systems claimed, not the scientific theories explaining how or why the invention functions.

To be clear, it is acknowledged that the foregoing hypotheses influenced the inventive process. But such hypotheses merely constituted motivational drivers for developing the methods and systems claimed. For whatever reason, the invention succeeds in enabling practitioners to penetrate the blood-brain barrier for drug-delivery and other purposes. That successful outcome holds true even if the above hypotheses are incorrect, imprecise, or incomplete.

In summary, five combinable and interchangeable processes have been disclosed. Those processes, as noted, involve the utilization of stretching means, inflaming means, overtaxing means, underoxygenating means, and immunocompromising means. Also disclosed are means for normalizing the permeability of cerebral capillaries after neurotherapeutics or neurodiagnostics are delivered. All such means/processes are effective in temporarily penetrating the blood-brain barrier and, regardless of the validity of the hypotheses motivating their conception, constitute the invention as claimed.

What is claimed is:

1. A drug-delivery system, said drug-delivery system comprising: an intravenously administered therapeutic or diagnostic agent; and means for delivering said therapeutic or diagnostic agent to an area of brain tissue.

2. The system of claim 1, further comprising: means for normalizing the permeability of cerebral capillaries after accomplishing delivery of said therapeutic or diagnostic agent.

3. A method and system for increasing the permeability of cerebral capillaries for therapeutic, diagnostic, or other purposes, said method and system comprising: implementing one or more processes selected from the group consisting of stretching means, inflaming means., overtaxing means, underoxygenating means, and immunocompromising means.

4. The method of claim 3, wherein said stretching means comprises the process of raising systole or diastole blood pressure and thereby inducing intracapillary hypertension.

5. The method of claim 3, wherein said stretching means comprises the administration of one or more vasoconstrictors.

6. The method of claim 3, wherein said stretching means comprises the administration of one · or more cardiostimulators.

7. The method of claim 3, wherein said inflaming means comprises the process of perturbing capillary tissue.

8. The method of claim 3, wherein said inflaming means comprises the electromagnetic bombardment of capillary tissue.

9. The method of claim 3, wherein said inflaming means comprises the sonic bombardment of brain tissue.

10. The method of claim 3, wherein said overtaxing means comprises the process of passing an overwhelming quantity of one or more diffusible substances through said capillaries.

11. The method of claim 3, wherein said overtaxing means comprises the administration of concentrated xenon gas.

12. The method of claim 3, wherein said underoxygenating means comprises the process of controlling and restricting oxygen intake using airway-interfacing equipment.

13. The method of claim 3, wherein said immunocompromising means comprises the process of diminishing the immune capabilities of the lymphatic system or local brain tissue.

14. The method of claim 3, wherein said immunocompromising means comprises the administration of one or more chemical, biological, or pharmacological immunosuppressants.

15. The method of claim 3, wherein said immunocompromising means comprises the administration of ionizing radiation.

16. A new use for xenon, said new use comprising the process of feeding xenon into the human respiratory system to increase the permeability of cerebral capillaries.

17. The new use for xenon of claim 16, wherein said xenon is administered in an amount volumetrically or gravimetrically greater than the amount of xenon present in ambient air.
